(19) United States
(12) Patent Application Publication
(10) Pub. No.: US 2024/0024228 A1
(43) Pub. Date: Jan. 25, 2024

(54) Apremilast Ophthalmic Compositions

(71) Applicant: APRAMITHA INNOVATIONS PRIVATE LIMITED, Hyderabad (IN)

(72) Inventors: Venkata Nookaraju Sreedharala, Hyderabad (IN); Srikanth Kalakoti, Hyderabad (IN); Ramdas Manakkote, Hyderabad (IN)

(21) Appl. No.: 18/256,733

(22) PCT Filed: Dec. 8, 2021
(86) PCT No.: PCT/IN2021/051146

§ 371 Date: Jun. 9, 2023

(30) Foreign Application Priority Data
Dec. 9, 2020 (IN) 202041053550
Dec. 10, 2020 (IN) 201941022869

(51) Int. Cl. A61K 9/00; A61K 31/4035; A61P 27/04; A61K 47/26; A61K 47/10; A61K 47/14

(52) U.S. Cl. CPC A61K 9/0048; A61K 31/4035; A61P 27/04; A61K 47/26; A61K 47/10; A61K 47/14

(57) ABSTRACT

The present invention relates to topical ophthalmic composition of Apremilast. The said ophthalmic composition has been designed for better penetration and reach to the target site for ophthalmic applications, thereby giving a better efficacious formulation. The present invention also relates to method of relieving the symptoms of dry eye by enhancing the tear secretion, reducing inflammation and moisturising the surface of eye thereby reducing the friction or irritation, as is generally caused due to various dry eye disorders. The presently proposed compositions are also proposed to be useful as delivery vehicles for ophthalmic drugs, and as wetting or lubrication substitute for the contact lens.

### CROSS REFERENCE

This application claims priority to Indian provisional patent application no. 202041053550, filed Dec. 9, 2020, and 201941022869, filed on Dec. 10, 2020, the contents of which are incorporated herein by reference in their entireties.

### FIELD OF INVENTION

The present invention relates to topical ophthalmic composition of Apremilast. The said ophthalmic composition has been designed for better penetration and reach to the target site for ophthalmic applications, thereby giving a better efficacious formulation. The present invention also relates to method of relieving the symptoms of dry eye by enhancing the tear secretion, reducing inflammation and moisturising the surface of eye thereby reducing the friction or irritation, as is generally caused due to various dry eye disorders. The presently proposed compositions are also proposed to be useful as delivery vehicles for ophthalmic drugs, and as wetting or lubrication substitute for the contact lens.

### BACKGROUND

Dry eye disorder (also known as DED) is a condition in which a person doesn't have enough quality tears to lubricate and nourish the eye, thereby leading to various symptoms like irritation, redness, swelling of eyes and sometimes corneal damage or vision loss. Dry eye is a common and often chronic problem, particularly in older adults.Dry eye syndrome (DES), also known as keratoconjunctivitissicca (KCS), is the similar condition of having dry eyes. Further, various other immune-mediated disorder cause inflammation of the ocular surface and lacrimal glands, collectively known as the “lacrimal functional unit” (LFU), which is also an intrinsic characteristic of both Sjogren's syndrome and non-Sjogren's syndrome—associated dry eye disorder.

The patho-physiology involved in dry eye disease is the inflammation of the LFU (lacrimal functional unit) occurs by activation of intracellular stress-associated mitogen-activated protein (MAP) kinase pathways that induce the production of pro-inflammatory cytokines such as IL-1 and TNF-a. These cytokines promote the activation and maturation of antigen-presenting cells (APCs) that subsequently migrate to draining lymphoid tissues and prime auto-reactive effector T cells. Adoptive transfer of CD4 [symbol] T helper (Th) cells from DED-induced donor mice to athymic (nude) recipient mice produces inflammation of the LFU similar to that observed in conventional DED, suggesting that DED is also a T-cell mediated autoimmune disorder.

It was estimated in few of the literatures that 6.8% of the US adult population were diagnosed with dry eye disorder, approximately around 16.4 million people. DED prevalence also increases with age (18-34 years: 2.7%; ≥75 years: 18.6%) and was higher among women (8.8%; approximately about 11.1 million) than men (4.5%; approximately about 5.3 million) and is ranged from 4.3% among men aged >50 years to 21.6% in men and women aged 48-91 years and 14.5% among those aged >21 years.

Various approaches like medications, artificial tears have been tried for dry eye disorder, such as anti-inflammatory and immunomodulatory medications, as corticosteroids and cyclosporine; they are utilized clinically in the treatment of DED. Corticosteroids (e.g., dexamethasone) are potent immunosuppressants that down regulate the activity of pro inflammatory molecules and lymphocytes.

The stated medications and corticosteroids are capable of ameliorating many cases of severe DED (dry eye disorders), unfortunately the side effects of prolonged corticosteroid use (e.g., as is known from cataract, glaucoma) generally make this an untenable choice. Also topical cyclosporine reduces DED severity by inhibiting the activity of T cells and promoting tear fluid secretion. Cyclosporine's efficacy in the treatment of DED is well established; however, many patients fail to respond favourably or adequately to cyclosporine therapy.

Medications that modulate various pro inflammatory molecules have shown promise in the treatment of experimental DED, but these medicines have not yet materialized in the clinical setting (Zahra Sadrai et al., Invest Ophthalmol Vis Sci. 2012 June; 53(7): 3584-3591). Most ophthalmologists agree that the currently available treatment modalities for moderate to severe DED are limited in both number and efficacy(Asbell P A et al., Ophthalmologist perceptions regarding treatment of moderate-to-severe dry eye: results of a physician survey. Eye Contact Lens. 2010; 36:33-38).

Thus in view of the shortcomings and harmful effects of medications like steroids and cyclosporine, the most suitable approach could be the evaluation of combinations with tear modulators, as human tears or artificial tears are necessary for maintaining the health of the front surface of the eye and for providing clear vision.

The present inventors have evaluated PDE 4 inhibitors for strategizing various ophthalmic compositions. The phosphodiesterase 4 (PDE4) enzymes regulate a host of biological processes by degrading the intracellular second messenger cAMP. PDE4 inhibitors have been intensively investigated as anti-inflammatory therapies because increases in cAMP levels are known to attenuate inflammatory responses in multiple cell types. Other agents that increase cAMP have been shown to induce tear secretion. Therefore, PDE4 inhibitors should serve the dual role of reducing inflammation and inducing tear secretion providing an effective treatment for dry eye.

Further the conventional ophthalmic PDE4 inhibitors like cilomilast and piclamilastare have been reported, however they are mostly eye drops. But the conventional ophthalmic drop's mean residence time (MRT) in the eye is too low due to constant lacrimal secretion which decreases the duration of action. Hence repeated administration is required. It is advantageous to formulate ophthalmic gel, or ophthalmic composition with higher consistency and viscosity which will have increased MRT than the solutions and thereby one can avoid repeated administration.

The inventors in the present application have evaluated Apremilast for dry eye disorder. Apremilast is a selective inhibitor of the enzyme phosphodiesterase 4 (PDE4) and inhibits spontaneous production of TNF-alpha from human rheumatoid synovial cells.Apremilast is chemically known as N-[2-[(1S)-1-(3-ethoxy-4-methoxyphenyl)-2-(methylsulfonyl)ethyl]-1,3-dioxo-2,3-dihydro-1H-isoindol-4-yl]acetamide. Apremilast is approved in United States of America and Europe, marketed by Celgene Corp as OTEZLA®. OTEZLA® tablets are supplied in 10, 20, and 30 mg strengths for oralAdministration. It is indicated for the treatment of active psoriatic arthritis and plaque psoriasis.

Further various literatures have been found for other topical applications of Apremilast, some of them have been briefed below as:

U.S. Pat. No. 6,872,382 B1 discloses method for the treatment of dry eye by administering to a mammal a composition comprising a pharmaceutically acceptable carrier and a pharmaceutically effective amount of one or more selective PDE-IV inhibitors.

WO2017/076987A1 discloses a method of treating or preventing a disease or disorder (Sjorgen's disorder) by administering a therapeutically or prophylactically effective amount of a solid solution or solid dispersion of Apremilast.

WO2017/216738 disclosed various topical pharmaceutical compositions of Apremilast comprising Apremilast pharmaceutically acceptable excipients for treatment of skin diseases.

US2019060221A1 discloses topical apremilast gels for psoriasis.

US2013/0172829A1 and US2013/0172790A1 discloses eye patches or strips affixed to the skin of upper and lower eyelids to deliver heat or other form of energy, pressure, drugs, moisture etc., for stimulating meibomian gland and treating dry eye disorder.

WO2014/018856A1 discloses composition and methods for treating dry eye disorder using progesterone on external surface of eye on facial region.

US2017/0348285A1 discloses ophthalmic combination composition of parasympathomymetic agent (like beta blockers) and anti-sympathetic agents (like cholinergics) for treatment of dry eye disorder

US20160101050A1 discloses ophthalmic nanoemulsions of cyclosporine for treatment of dry eye disorder.

Although there are various topical compositions available in the literature, only few of them were used for dry eye disease and even a very few talk about Apremilast or any suitable phosphodiesterase inhibitors for ophthalmic applications. Therefore the state of the art clearly concludes that ophthalmic composition of Apremilast and PDE4 inhibitors are very few and rare. The possible reason behind this is the poor aqueous solubility of PDE4 inhibitors, specifically Apremilast, in water and many other solvents, which makes it difficult for formulators to envisage a suitable ophthalmic composition of Apremilast which can be effective for treatment of dry eye disorder and other similar conditions.

A drawback of above prior art compositions and routine conventional topical and ophthalmic formulations is that their active component acts for a short duration of time, thereby requiring frequent re-application which leads to a negative impact on treatment compliance and quality of life of the patient. Also such compositions are complex and require lot of critical manufacturing aspects, thus they are costlier. Consequently, it is sometimes difficult for patients to maintain regular applications of these medications. Moreover, abrupt withdrawal of Apremilast particularly in DED, may cause an aggressive recurrence of the condition or severe impact on overall patient health.

Despite the presence of various ophthalmic compositions, there is need in the art for a stable and highly effective, cost-effective composition which can provide controlled/sustained release of the drug and is consistent and non-irritant over conventional ophthalmic compositions and shall be long acting, which could also be used in chronic cases of DED.

As already discussed and known in the literature that due to the insolubility of Apremilast itself in water and minimal solubility in polar solvents, it is challenging to formulate a proper topical composition of Apremilast having better penetrating capabilities and providing an effective concentration of Apremilast at the site of action for a longer period of time to give enhanced therapeutic results without any irritation to eye. Hence, there remains an unmet need to develop suitable and easy to apply formulations like ophthalmic drops, solution, gels and clear dispersions of Apremilast, which can give better penetration and efficacy with reduced dosage and frequency of administration.

The present application envisages formulation of various promising ophthalmic compositions, which are not only effective in treating DED, are also suitable for chronic use and can be used in conjugation with other dry eye therapies. The inventors of the present invention found that ophthalmic composition of Apremilast with specific combination of solubilizers or surfactants, tear stimulants, permeation enhancer, lubricants and suitable ophthalmic adjuvant can significantly achieve an enhanced ocular penetration, thus better efficacy with site specific drug delivery and provides a much better treatment approach. Also the key aspect for proposed ophthalmic composition is penetration or diffusion into the corneal layers for better efficacy and still non-irritant to eye with longer mean residence time (MRT).

### SUMMARY OF THE INVENTION

The present invention provides ophthalmic compositions of Apremilast designed for better penetration and reach to the target site for ophthalmic applications.

A further object of the present invention is to provide methods for relieving the symptoms of dry eye by enhancing tear secretion and moisturising the surface of eye thereby reducing the friction or irritation caused in case of various dry eye disorders.

Another object of the present invention is to provide additive compositions that can be used as delivery vehicles for ophthalmic drugs, and as wetting or lubrication substitute for contact lens.

An yet another object of the present invention is to provide an ophthalmic formulation in the form of gel, solution, drops, emulsions, emulgels and dispersions for prolonged release of Apremilast and alleviating the symptoms of DED in chronic therapy.

### BRIEF DESCRIPTION OF DRAWINGS

FIG. 1: Polarizing light microscopy (PLM) images of Apremilast Ophthalmic Emulsion Example-4 at stage M1 (50X).

FIG. 2: Polarizing light microscopy (PLM) images of Apremilast Ophthalmic Emulsion Example-4 at stage M2 (50X).

FIG. 3: Polarizing light microscopy (PLM) images of Apremilast Ophthalmic Emulsion Example-4 at stage M3 (50X).

FIG. 4: Polarizing light microscopy (PLM) images of Apremilast Ophthalmic Emulsion Example-4 at stage M1 (100X).

FIG. 5: Polarizing light microscopy (PLM) images of Apremilast Ophthalmic Emulsion Example-4 at stage M2 (100X).

FIG. 6: Polarizing light microscopy (PLM) images of Apremilast Ophthalmic Emulsion Example-4 at stage M3 (100X).

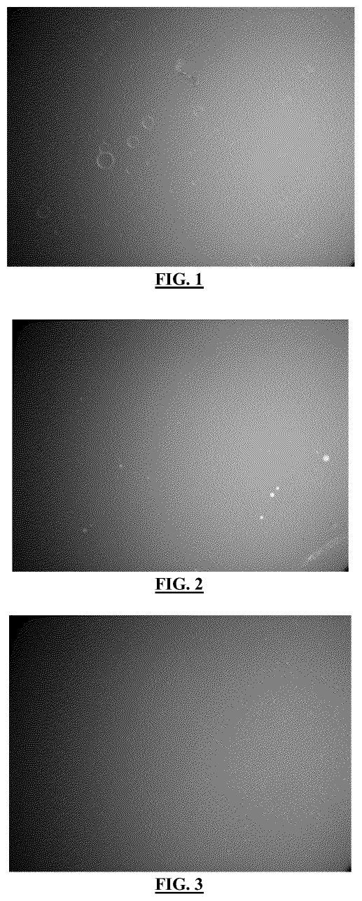

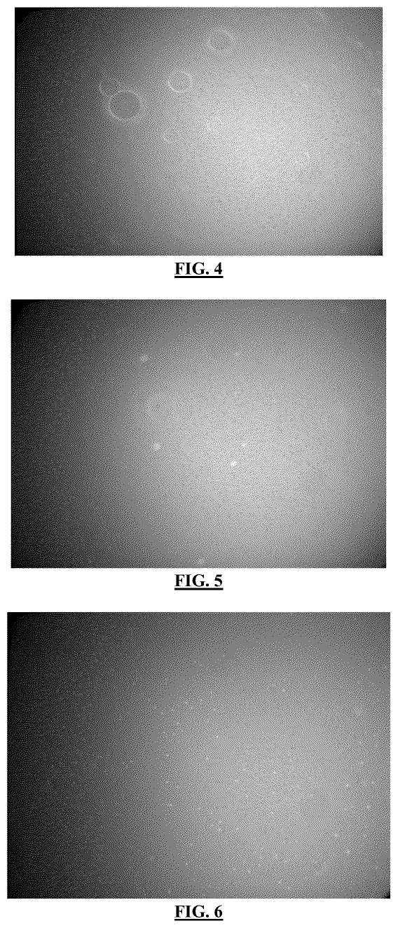

### DETAILED DESCRIPTION

The present disclosure is herein described in detail with reference to embodiments, which form a part here. Other embodiments may be used and/or other changes may be made without departing from the spirit or scope of the present disclosure. The illustrative embodiments described in the detailed description are not meant to be limiting of the subject matter presented here.

The present invention pertains to ophthalmic compositions of Apremilast with suitable solubilizers and tear stimulants for treatment of various symptoms of dry eye disorder. The composition of the present invention envisages a careful selection of specific excipients in specific ratios and combinations, for completely solubilizing the active agent (Apremilast) which has poor aqueous solubility and forming a longer acting, better penetrating consistent ophthalmic composition.

In another embodiment, the present invention provides an ophthalmic gel, solution, drop, emulsion and emulgel of Apremilast with at least one surfactant and having enhanced ocular delivery of drug with simultaneous tear stimulation, thereby providing compositions with better patient compliance and consistency.

In yet few other preferred embodiments, the present invention provides longer acting controlled release ophthalmic compositions of Apremilast comprising specific quantities of at least one surfactant, at least one permeation enhancer, at least on solvent, at least one emulsifier and a pH adjusting agent to maintain a pH in the suitable range for better stability and shelf life of the formulation.

In another embodiment the present invention relates to ophthalmic composition of Apremilast for treatment of dry eye disorder (DED), Dry eye syndrome (DES), Keratoconjunctivitis sicca (KCS), Sjogren's syndrome or any other related conditions in which a person doesn't have enough quality tears to lubricate and nourish the eyes, thereby leading to various symptoms like irritation, redness, inflammation and swelling of eyes.

In another embodiment of the invention is provides an ophthalmic composition of Apremilast with suitable excipients selected from surfactants, solubilizers, solvents, co-solvents, carrier, oil vehicles, preservatives, antioxidants, gelling agents, lubricants, humectants, moisturisers, lubricants, buffers and aqueous bases, or combinations thereof.

In a further embodiment the present invention provides an ophthalmic composition of Apremilast where drug is dissolved in surfactant or a mixture of surfactant, by heating or by any suitable method of dissolving known to a person skilled in the art. Non-limiting methods for dissolving the drug includes sonication, vortexing or shaking.

In a further embodiment the present invention provides an ophthalmic composition of Apremilast where drug is dissolved in a combination of surfactant or a mixture of surfactant, and suitable permeation enhancers, by heating or by any suitable method of dissolving known to a person skilled in the art. Non-limiting methods for dissolving the drug includes sonication, vortexing or shaking.

In a further embodiment the present invention provides an ophthalmic composition of Apremilast and suitable moisturiser or humectant, where the components are dissolved in a surfactant or a mixture of surfactant, by heating or by any suitable method of dissolvingknown to a person skilled in the art. Non-limiting methods for dissolving the drug includes sonication, vortexing or shaking.

In a further embodiment the present invention provides an ophthalmic composition of Apremilast with suitable permeation enhancer, wherein the said components in the composition in-fact act synergistically, leading to an enhanced tearing of eye thereby relieving the symptoms of DED.

In a further embodiment the present invention provides an ophthalmic composition of Apremilast or any suitable PDE4 inhibitor with suitable lubricant or humectant, wherein the said components in the composition in-fact act synergistically, leading to an enhanced tearing of eye thereby relieving the symptoms of DED.

In a further embodiment the lubricant or humectant used in present invention, provide dual role of tear stimulation and lubrication of eye thereby promoting tearing of eye and provide relief from DED symptoms like dryness, irritation, swelling and itching of eye.

In a further embodiment the present invention provides an ophthalmic composition which can be easily added as an adjuvant or additive to any suitable marketed composition for promoting the tearing of eye or relieving the symptoms of DED.

In a further related embodiment, the present invention has been developed to prolong the MRT of the composition and for enhancing the diffusion rate and penetration of the drug through ocular layers.

In a most preferred embodiment of the invention, the ophthalmic composition comprises of Apremilast or its pharmaceutically acceptable salt and a surfactant or a mixture of two or more surfactant in suitable ratio, to give maximum solubilization of the drug and to promote stability and integrity of the composition.

In another most preferred embodiment of the invention, the ophthalmic composition comprises of Apremilast or its pharmaceutically acceptable salt and a solubilizer or a mixture of two or more solubilizer in suitable ratio, to give maximum solubilization of the drug and to promote stability and integrity of the composition.

Unless otherwise recited or required by the context, percent and “%” refer to percent by weight.

Ophthalmic compositions of the invention include Apremilast in the concentration of 0.001% to 0.9% by weight, preferably 0.03%-0.09% w/w.

Ophthalmic compositions of the invention have a pH in the physiological range between 3.0-9.0, preferably in the range of 4.0-6.0.

In a further embodiment the composition of the present invention comprises Apremilast in the range of 0.001-0.9% w/w, surfactant/solubilizer in the range of 0.2-10% w/w, humectant in the range of 0.05-5% w/w, lubricant in the range of 0.05-15% w/w, preservative in the range of w/w, aqueous base 10-99% and pH adjusting agent quantity sufficient.

Suitable surfactants/solubilizers include an alkyl polyglycol ether, an alkyl polyglycol ester, an ethoxylated alcohol, a polyoxyethylene sorbitan fatty acid ester, a polyoxyethylene fatty acid ester, an ionic or non-ionic surfactant, a hydrogenated oil/polyoxyethylene glycol adduct containing from 25 to 60 ethoxy groups a castor oil/polyoxyethylene glycol adduct containing from 25 to 45 ethoxy groups, a sorbitan fatty acid ester (for example Span 20 or Span 80), a block copolymer of ethylene oxide and propylene oxide (for example Pluronic L121 or Pluronic F68), or a mixture thereof.

Examples of classes of surfactants/solubilizers which are particularly useful in this invention include: surfactants/solubilizers include poloxamers, tyloxapol, polysorbates macrogolglycerol ricinoleate, sorbitan esters, and mixtures of two or more thereof. The surfactant may be polyoxyethylene sorbitan fatty acid ester (e.g., Polysorbate 20, Polysorbate 80, and so on), polyoxyethylene-polyoxypropylene block copolymer (e.g., Poloxamer 188.™. and so on), macrogolglycerol ricinoleate (Kolliphor® EL), or a mixture thereof, preferably polyoxyethylene (80) sorbitan monolaurate (Polysorbate 80) or polyoxyethylene (80) sorbitan monooleate (Polysorbate 80), more preferably polyoxyethylene (80) sorbitan monolaurate.

One or more of each type of surfactant may be present in the composition. Additionally or alternatively mixtures of different types of surfactant may be present in the composition. One of the reasons these surfactants are particularly preferred is because of their low irritation potential. The present inventors have also surprisingly found that the above surfactants provide good shear stability over other known surfactants.

In another embodiment the composition of the present invention comprises Apremilast in the range of 0.001-0.9% w/w, surfactant/solubilizer in the range of 0.2-10% w/w, co-solvent in the range of 0.05-5% w/w, permeation enhancer in the range of 0.05-5% w/w, gelling agent in the range of 0.05-5% w/w, preservative in the range of 0.01-4% w/w, aqueous basein the range of 10-99% and pH adjusting agent quantity sufficient.

In another embodiment the composition of the present invention comprises Apremilast in the range of 0.001-0.9% w/w, surfactant/solubilizer in the range of 0.2-10% w/w, co-solvent in the range of 0.05-5% w/w, permeation enhancer in the range of 0.05-5% w/w, gelling agent in the range of 0.05-5% w/w, preservative in the range of 0.01-4% w/w, an oil phase in the range of 1-20% and an aqueous base in the range of 20-99% and pH adjusting agent quantity sufficient.

In another embodiment the composition of the present invention comprises Apremilast in the range of 0.001-0.9% w/w, oil vehicle in the range of 1.5-10% w/w, surfactant/solubilizer in the range of 0.2-10% w/w, co-solvent in the range of 0.05-5% w/w, permeation enhancer in the range of 0.5-5% w/w, preservative in the range of 0.01-4% w/w, aqueous base 10-99% and pH adjusting agent quantity sufficient.

In another embodiment the present invention provides an ophthalmic emulsion of Apremilast for treating dry eye disorder, wherein the ophthalmic emulsion comprises of Apremilast in an amount of 0.001-0.9% w/w, a permeation enhancer in an amount of 0.25-10% w/w and at least one lubricant in an amount of 0.05-15% w/w.

In yet another embodiment the present invention provides an ophthalmic gel of Apremilast for treating dry eye disorder, wherein the ophthalmic gel comprises of Apremilast in an amount of w/w, at least one permeation enhancer in an amount of 0.05-5% w/w and a pharmaceutically acceptable gelling agent in an amount of 0.05-5% w/w.

In a further embodiment the present invention provides an ophthalmic composition of Apremilast for treating dry eye disorder, wherein the topical composition comprises of Apremilast in an amount of 0.001-0.9% w/w, at least one tear stimulator in an amount of 0.05-10% w/w and at least one permeation enhancer in an amount of 0.05-5% w/w.

In yet another related embodiment of the present invention the tear stimulator is not a drug; it is lubricant or any other solubilizer or permeation enhancer and the permeation enhancer is the co-solubilizer.

Suitable permeation enhancers include propylene glycol. Other suitable ocular permeation enhancers may be selected from one or more and mixtures of the following non-exhaustive list: Surfactants: Sorbitan glycerides (SPAN 20, polyoxyethylene sorbitan glycerides 40, 85); (TWEEN 20, 40, 81); polyethylene glycol 1000 stearate (Aptet 100); G 1045; polyoxyethylene stearyl ethers (BRIJ 23, 35, 48, 58, 78, 98); polyoxyethylene stearate (MYRJ S40, S50); polyoxyethylene glycol adduct containing from 25 to 60 ethoxy groups (CREMOPHOR E L/KOLLIPHOR E L); BL-9; polyoxyethylene p-(tetramethylbutyl)phenyl ether (TRITON X-100); saponin Bile acids and bile salts: Deoxycholic acid; taurocholic acid; taurodeoxycholic acid; urodeoxycholic acid; tauroursodeoxycholic acid; sodium cholate; sodium glycocholate Fatty acids: capric acid Preservatives: benzalkonium chloride; benzododecinium bromide; chlorhexidine digluconate; benzyl alcohol; chlorbutanol; 2-phenyl ethanol; paraben; propyl paraben Chelating agents: EDTA Others: 1-dodecylazacycloheptan-2-one (Azone); hexamethylene lauramide; hexamethyleneoctanamide; decylmethylsulfoxide; PHARMASOLVE (N-methyl pyrollidone); GELUCIRE 44/14 (Lauroyl macrogol-32 glycerides); borneol; dimethyl sulphoxide; sodium fusidate; decamethonium bromate; cetyl pyridinium chloride; a-amino acids; cyclodextrins; medium chain monoglycerides; cetrimide; cytochalasins.

In yet another preferred embodiment the ratio of drug to surfactants/solubilizers in the composition is ranging from 1:100 to 100:1 or 1:50 to 50:1 or 1:5 to 10:50. The solubilizing agent could be single or multiple surfactant or solubilizers.

In yet another preferred embodiment the ratio of drug to tear stimulating agents in the composition is ranging from 1:100 to 100:1 or 1:50 to 50:1 or 1:5 to 10:50. The tear stimulators agent could be one or more than one tear stimulators.

In another preferred embodiment the process for preparation of the ophthalmic composition characteristically involves drug addition to non-aqueous phase.

Non-limiting lists of the excipients that can be used in the composition are:

Preferred non-limiting examples of excipients includes pharmaceutical surfactant, Solubilizers, solvents, co-solvents, permeation enhancers, moisturisers, humectant, tear stimulators, gelling agents, emulsifiers, oil phase carriers and vehicles, lubricants, buffers, preservatives, antioxidants, aqueous and non-aqueous bases, pH adjusting agents, inorganic salts and other ingredients. Also the components/excipients as embodied in the present composition may have more than one application.

In an embodiment of the invention, ophthalmic composition of present invention has pH adjusting agents that help in adjusting the pH of the composition between pH of about 3 to about 9, preferably 4 to 7 to provide a stable and non-irritating composition.

The compositions of the present invention can also be used in combination with additional pharmaceutically active compounds. Such compounds include, but are not limited to, glaucoma therapeutics, pain relievers, anti-inflammatory and anti-allergy medications, and anti-microbials. More specific examples of pharmaceutically active compounds include betaxolol, timolol, pilocarpine, carbonic anhydrase inhibitors and prostglandins; dopaminergic antagonists; post-surgical antihypertensive agents, such as para-amino clonidine (apraclonidine); anti-infectives such as ciprofloxacin, moxifloxacin, and tobramycin; non-steroidal and steroidal anti-inflammatories, such as naproxen, diclofenac, nepafenac, suprofen, ketorolac, tetrahydrocortisol and dexamethasone; other PDE4 inhibitors; and anti-allergy medications such as H1/H4 inhibitors, H4 inhibitors, olopatadine or mixtures thereof.

It is also contemplated that the concentrations of the ingredients comprising the formulations of the present invention can vary. A person of ordinary skill in the art would understand that the concentrations can vary depending on the addition, substitution, and/or subtraction of ingredients in a given formulation.

In certain embodiments, the compositions of the present invention is in the form of eye-drop solution, eye wash solution, contact lens lubricating and/or rewetting solution, spray, mist or any other manner of administering a composition to the eye.

In particular embodiments, the composition of the present invention are formulated for administration at any frequency of administration, including once a week, once every five days, once every three days, once every two days, twice a day, three times a day, four times a day, five times a day, six times a day, eight times a day, every hour, or greater frequency. Such dosing frequency is also maintained for a varying duration of time depending on the therapeutic.

### EXAMPLES

The following examples which include preferred embodiments will serve to illustrate the practice of this invention, it being understood that the particulars shown are by way of example and for purposes of illustrative discussion of preferred embodiments of the present invention only and are presented in the cause of proving what is believed to be the most useful and readily understood description of formulation procedures as well as of the principles and conceptual aspects of the invention. It will be evident to those skilled in the art that the invention is not limited to the details of the following illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### Example 1: Solubility Study

The solubility of Apremilast was evaluated in various surfactants and their combinations:

Procedure Followed:

About 50 mg of Apremilast (0.05% w/w) was weighed and dissolved in surfactant and or combination of surfactants at different ratios by heating at a temperature below 85° C. After complete solubilization of the Apremilast in the solubilizer matrix, purified water (q.s to 100%) was added, vortexed for 2 minutes and kept aside.

The Apremilast solutions were observed at different time intervals (24 hrs to 1 week) for precipitation of the drug. In the present solubility study, the concentration of Apremilast evaluated for solubility was in the range of 0.001% to 0.9%; and the solubilizers or surfactants that were evaluated in the present study includes Tween 80, PEG 400, Polaxomer 188, Kolliphor E L and few others in the ranges of 0.1 to 10%.

Observations:

In the study it was found that Apremilast behaved differently in different surfactants and the optimum solubility for preparation of an ophthalmic non-irritating composition is very critical. The study findings were presented as below:
Apremilast has good solubility in Tween 80 and kolliphor E. L. Also it has shown good solubility in combination of surfactants (Tween 80 and Kolliphore E L) with PEG400.
Precipitation of Apremilast was not observed with 2-8% Tween80 and 2-8% kolliphor E. L. Below to this concentration, API was precipitated out from drug solution. 4% w/w of these surfactants was found to be sufficient to stabilise 0.001-0.9% w/w of Apremilast.
Also the Apremilast precipitation was observed with 5% PEG 400. So PEG400 alone is not sufficient for stabilization of API. Whereas along with combination of Tween 80/Kolliphore E L, API was stabilized.
The combination of Tween 80 and kolliphor E. L gives better stability of API by preventing precipitation, thereby giving clear solution, suitable for ophthalmic compositions.

Based on the above solubility study, various ophthalmic compositions in form of solutions, drops, gels, emulsions and emulgels were optimized and evaluated for further parameters like irritation, stability, penetration, efficacy and MRT.

The non-limiting examples of the various ophthalmic compositions are as follows:

### Example 2: Apremilast Ophthalmic Solution/Drops

| Component | % w/w |
|---|---|
| Apremilast | 0.02 |
| Tween | 4 |
| Glycerin | 2 |
| Propylene glycol | 10 |
| Methyl paraben | 0.05 |
| propyl paraben | 0.04 |
| Purified water | 83.8 |
| NaOH Solution | q.s. |
| Total | 100 |

Brief Manufacturing Process:
1. Apremilast was dissolved in suitable surfactant or solubilizer by heating at a temperature below 85° C. After solubilization of API suitable preservatives were dissolved under the similar conditions.
2. The resulting solution of step 1) was cooled to room temperature and suitable humectant and lubricants were added.
3. The solution form step 2) was added to purified water under continuous stirring
4. Finally the pH of the solution obtained from step 3) was adjusted with NaOH solution to a pH in the range of 6.5-7.5.

### Example 3: Apremilast Ophthalmic Gel

| Component | % w/w |
|---|---|
| Apremilast | 0.005 |
| Tween | 4.045 |
| Glycerin | 1 |
| Propylene glycol | 1 |
| Carbopol | 0.5 |
| Methyl paraben | 0.05 |
| propyl paraben | 0.01 |
| Purified water | 93.4 |
| NaOH Solution | q.s. |
| Total | 100 |

Brief Manufacturing Process:
1. Apremilast was dissolved in suitable surfactant or solubilizer by heating at a temperature below 85° C. After solubilization of API suitable preservatives were dissolved under the similar conditions.
2. The resulting solution of step 1) was cooled to room temperature and suitable permeation enhancer and co-solvent were added under stirring.
3. A dispersion of suitable gelling agent was prepared by dispersing the gelling agent in purified water.
4. The dispersion obtained in step 3) was added to the solution from step 2) under continuous stirring and the pH was adjusted with about NaOH solution to pH 6.5-7.5.

### Example 4: Apremilast Ophthalmic Emulsion

| Component | % w/w |
|---|---|
| Apremilast | 0.05 |
| Kolliphor EL | 3 |
| Tween | 4 |
| Glycerin | 1 |
| Propylene glycol | 1 |
| Methyl paraben | 0.05 |
| propyl paraben | 0.01 |
| Purified water | 90.8 |
| NaOH Solution | q.s. |
| Total | 100 |

Brief Manufacturing Process
1. Apremilast was dissolved in suitable oil and surfactant or solubilizer by heating at a temperature below 85° C. After solubilization of API suitable preservatives were dissolved under the similar conditions.
2. The resulting solution of step 1) was cooled to room temperature and suitable permeation enhancer and co-solvent were added under stirring.
3. Purified water was added to step 2) solution under continuous stirring.
4. pH of step 3 resultant emulsion obtained in step 3) was adjusted to pH 6.5-7.5, with about NAOH solution.
5. The consistency of the formulation did not deteriorate on storage and was found to be sufficiently stable;
6. No crystals were observed under PLM.

All the above compositions were evaluated for stable and consistency. The above compositions were further optimized to give better patient compliance and evaluated on animal models for various parameters like redness of eye, repeated pawing of test animals, rubbing of eye by test subjects, number of blinking and tearing.

### Example 5: Stability of Compositions of this Invention

Stability of the emulsion obtained in Example-4 was assessed by measuring physicochemical parameters average globule size, polydispersibility and zeta potential after sterilization by autoclave or filtration and during an accelerated stability test at 80° C.

Results:

TABLE 1
Average globule size and Polydispersibility Index

| | | | |
|---|---|---|---|
| | | Z-Average | Polydispersible index |
| S. No | Parameter | (d · nm) | (PDI) |

| | |
|---|---|
| Stage | Mixing with mechanical stirrer (S1) |

| | | | |
|---|---|---|---|
| 1 | Trial -1 | 349.5 | 0.482 |
| 2 | Trial -2 | 298.7 | 0.536 |
| 3 | Trial -3 | 260.3 | 0.469 |
| | Average | 302.8 | 0.496 |

| | |
|---|---|
| Stage | High shear mixing for 10 min (R1) |

| | | | |
|---|---|---|---|
| 1 | Trial -1 | 250.9 | 0.517 |
| 2 | Trial -2 | 248.6 | 0.530 |
| 3 | Trial -3 | 250.8 | 0.477 |
| | Average | 250.1 | 0.508 |

| | |
|---|---|
| Stage | High shear mixing for 20 min (R2) |

| | | | |
|---|---|---|---|
| 1 | Trial -1 | 269.0 | 0.536 |
| 2 | Trial -2 | 242.8 | 0.440 |
| 3 | Trial -3 | 239.1 | 0.336 |
| | Average | 250.3 | 0.437 |

| | |
|---|---|
| Stage | High shear mixing for 30 min (R3) |

| | | | |
|---|---|---|---|
| 1 | Trial -1 | 409.9 | 0.499 |
| 2 | Trial -2 | 276 | 0.507 |
| 3 | Trial -3 | 258.4 | 0.451 |
| | Average | 314.8 | 0.486 |

| | |
|---|---|
| Stage | Microfluidization for 1 cycle (M1) |

| | | | |
|---|---|---|---|
| 1 | Trial -1 | 143.4 | 0.201 |
| 2 | Trial -2 | 144.2 | 0.225 |
| 3 | Trial -3 | 146.1 | 0.206 |
| | Average | 144.6 | 0.211 |

| | |
|---|---|
| Stage | Microfluidization for 2 cycles (M2) |

| | | | |
|---|---|---|---|
| 1 | Trial -1 | 138.6 | 0.194 |
| 2 | Trial -2 | 138.8 | 0.198 |
| 3 | Trial -3 | 140.5 | 0.188 |
| | Average | 139.3 | 0.193 |

| | |
|---|---|
| Stage | Microfluidization for 3 cycles (M3) |

| | | | |
|---|---|---|---|
| 1 | Trial -1 | 183.8 | 0.295 |
| 2 | Trial -2 | 180.2 | 0.304 |
| 3 | Trial -3 | 177.5 | 0.308 |
| | Average | 180.5 | 0.302 |

TABLE 2
Zetapotential

| S. No | Stage | Trial-1 | Trial-2 | Trial-3 | Average |
|---|---|---|---|---|---|
| 1 | S1 | −0.092 | −0.648 | −0.914 | −0.55 |
| 2 | R1 | −0.537 | −0.154 | 0.458 | −0.08 |
| 3 | R2 | 0.130 | 0.336 | 0.849 | 0.44 |
| 4 | R3 | −0.294 | −0.089 | −0.482 | −0.29 |
| 5 | M1 | −0.030 | 0.188 | −0.589 | −0.14 |
| 6 | M2 | −0.276 | −0.346 | −0.400 | −0.34 |
| 7 | M3 | −0.663 | −0.339 | −0.217 | −0.41 |

Conclusion:

The consistency of the formulation did not deteriorate on storage and was found to be sufficiently stable.

### Example-6: Acute Eye Irritation Study of Apremilast Ophthalmic Solution and Gel in New Zealand White Rabbit

Test samples:
1. Apremilast Ophthalmic Gel Composition (Example 3)
2. Apremilast Ophthalmic Solution Composition (Example 2)

Study Design:

| | | | No of |
|---|---|---|---|
| Group | | | animals/ |
| No. | Treatment | Dosage | group |
| G1 | Phosphate buffer solution (PBS) | 0.1 ml, once daily | 2 |
| G2 | Placebo of Apremilast ophthalmic | 0.1 ml, once daily | 2 |
| | solution | | |
| G3 | Placebo of Apremilast ophthalmic | 0.1 g, once daily | 2 |
| | gel | | |
| G4 | Apremilast ophthalmic solution | 0.1 ml, once daily | 2 |
| G5 | Apremilast ophthalmic gel | 0.1 g, once daily | 2 |

Method:

After one week of acclimatization, the animals (Rabbits) were randomized into 5 groups. Group 1 animals were treated with PBS in right of the animals and were considered as control group. Group 2-Group 5 rabbits are treated with the respective test samples as described in study design.

After treating the animals with the specified dose, the animals were observed for three days. On the day of treatment, the animals were observed three times (Morning just after treatment, noon and evening) in a day and the remaining two days animals were observed for two times (Morning and evening) in a day. The observation was carried out for three min. During the three minutes of observation different clinical parameters such as redness, repeated pawing, rubbing, no of blinks and tearing was recorded. The results are tabulated as follows.

Test Results:

| | | Obser- | | | Rub- | | |
|---|---|---|---|---|---|---|---|
| | | vation | Red- | Repeated | bing | No of | Tear- |
| Day | Group | time | ness | pawing | eye | Blinks | ing |
| 1 | G1 | 3 min | No | No | No | 0 | No |
| | | | No | No | No | 0 | No |
| | | | No | No | No | 0 | No |
| 2 | | | No | No | No | 0 | No |
| | | | No | No | No | 0 | No |
| 3 | | | No | No | No | 0 | No |
| | | | No | No | No | 0 | No |
| 1 | G2 | 3 min | No | No | No | 0 | No |
| | | | No | No | No | 0 | No |
| | | | No | No | No | 0 | No |
| 2 | | | No | No | No | 0 | No |
| | | | No | No | No | 0 | No |
| 3 | | | No | No | No | 0 | No |
| | | | No | No | No | 0 | No |
| 1 | G3 | 3 min | No | No | No | 0 | No |
| | | | No | No | No | 0 | No |
| | | | No | No | No | 0 | No |
| 2 | | | No | No | No | 0 | No |
| | | | No | No | No | 0 | No |
| 3 | | | No | No | No | 0 | No |
| | | | No | No | No | 0 | No |
| 1 | G4 | 3 min | No | No | No | 8 | No |
| | | | No | No | No | 1 | No |
| | | | No | No | No | 0 | No |
| 2 | | | No | No | No | 0 | No |
| | | | No | No | No | 0 | No |
| 3 | | | No | No | No | 0 | No |
| | | | No | No | No | 0 | No |
| 1 | G5 | 3 min | No | No | No | 0 | No |
| | | | No | No | No | 0 | No |
| | | | No | No | No | 0 | No |
| 2 | | | No | No | No | 0 | No |
| | | | No | No | No | 0 | No |
| 3 | | | No | No | No | 0 | No |
| | | | No | No | No | 0 | No |

Conclusion:

The study results substantiated that the test samples are non-irritant to the eye.

### Example-7: In-Vivo Efficacy Study of Apremilast Ophthalmic Solution and Gel in Induced Dry Eye In Balb/C Mice

Test System:
Species: Mice
Strain: Balb/c
Sex: Male
Age: 8-10 weeks

Test samples:
1. Apremilast Ophthalmic Gel Composition (Example 3)
2. Apremilast Ophthalmic Solution Composition (Example 2)

Allocation of Groups:

| | | | No of |
|---|---|---|---|
| Group | | | animals/ |
| No. | Treatment | Dosage | group |
| G1 | Phosphate buffer solution (PBS) | 0.1 ml, once daily | 8 |
| G2 | Placebo of Apremilast ophthalmic | 0.1 ml, once daily | 8 |
| | solution | | |
| G3 | Placebo of Apremilast ophthalmic | 0.1 g, once daily | 8 |
| | gel | | |
| G4 | Apremilast ophthalmic solution | 0.1 ml, once daily | 8 |
| G5 | Apremilast ophthalmic gel | 0.1 g, once daily | 8 |

Method:
After one week of acclimatization, the animals (Mice) were randomized based on body weight divided into 5 groups.
Group 1 served as normal control mice and was be treated with PBS in the right eyes as the PBS control group. The ophthalmic preparation was adjusted to iso-osmia before used.
Group 2-Group 5 the right eyes of mice were treated with twice-daily (9 AM, 9 PM) topical administration of 5 μl of 0.2% BAC for 7 days as the BAC-induced dry eye.
Group 3-Group 5 was topically treated with the respective test samples as described in study design from day 1 to day 7.
Tear volume was measured using ZONEQUICK cotton threads on days 0, 1, 4 and 7.
Evaluation of inflammation: Inflammatory response was evaluated by slit lamp on days 0, 1, 4, 7.
On day 7, all mice were sacrificed, and the ocular global tissues carefully dissected, harvested and fixed in formalin for histological analysis.

Conclusion:

Administration of the proposed formulation, twice daily, shows clinical efficacy in decreasing corneal epithelial defects. Histopathological investigation of eye further conforms the above observation.

Having now fully described the invention, it will be apparent to one of ordinary skill in the art that many changes and modifications can be made thereto without departing from the spirit or scope of the invention as set forth herein.

1. A stable ophthalmic composition for topical administration, the composition comprising:
Apremilast; and
a surfactant or solubilizer, wherein:
the amount of Apremilast is 0.001-0.9% w/w,
the pH of the composition is 3.0-9.0, and
the stable ophthalmic composition is effective for the treatment of inflammatory disorders of the ophthalmic system.

2. (canceled)

3. The stable ophthalmic composition of claim 1, wherein the pH of the composition is in a range of 4.0-7.0.

4. (canceled)

5. The stable ophthalmic composition of claim 1, wherein the surfactant or solubilizer is selected from the group consisting of anionic or cationic or non-ionic, or a mixture thereof.

6. (canceled)

7. The stable ophthalmic composition of claim 1, wherein the composition further comprises a 0.05-5% w/w of a permeation enhancer.

8. A stable ophthalmic composition for treatment of symptoms of inflammatory disorders of the ophthalmic system, comprising:
i. Apremilast in a range of 0.001-0.9% w/w;
ii. at least one surfactant or solubilizer in a range of 0.2-10% w/w; and
iii. a buffer,
wherein a pH range of the ophthalmic composition is between 4 and 7, and wherein the ophthalmic composition is transparent and free of precipitation layer.

9. (canceled)

10. The stable ophthalmic composition of claim 8, wherein the composition further comprises at least one additive selected from the group consisting of a tear stimulator, a permeation enhancer, and a lubricating agent.

11. A method of treating symptoms of dry eye, comprising administering an ophthalmic composition to an eye, wherein the ophthalmic composition includes:
i. 0.001-0.9% w/w of Apremilast; and
ii. 0.2-10% w/w of at least one surfactant or solubilizer.

12. The method of claim 11, wherein the surfactant or solubilizer is selected from the group consisting of anionic or cationic or non-ionic, or a mixture thereof.

13. The method of claim 11, wherein the ophthalmic composition has a pH in a range of 3.0-9.0.

14. The method of claim 11, wherein the ophthalmic composition further comprises at least one additive selected from the group consisting of a tear stimulator, a permeation enhancer, and a lubricating agent.

15. The stable ophthalmic composition of claim 1, wherein the composition is a solution.

16. The stable ophthalmic composition of claim 1, wherein the composition is a gel.

17. The stable ophthalmic composition of claim 1, wherein the composition is an emulsion.

18. The stable ophthalmic composition of claim 8, wherein the composition is a solution.

19. The stable ophthalmic composition of claim 8, wherein the composition is a gel.

20. The stable ophthalmic composition of claim 8, wherein the composition is an emulsion.

21. The stable ophthalmic composition of claim 1, wherein the surfactant or solubilizer is present in a range of 0.2 to 10% w/w.

22. The stable ophthalmic composition of claim 8, wherein the surfactant or solubilizer is selected from the group consisting of anionic or cationic or non-ionic, or a mixture thereof.
